(19) United States
(12) Patent Application Publication
(10) Pub. No.: US 2025/0288208 A1
(43) Pub. Date: Sep. 18, 2025

(54) METHOD AND SYSTEM FOR VISUALIZATION OF FAINT FLUORESCENCE IN SURGERY, SOFTWARE PROGRAM

(71) Applicant: Quest Photonic Devices B.V., SR Wieringerwerf (NL)

(72) Inventors: Thomas KOOPMAN, WR Amsterdam (NL); Richelle Johanna Maria HOVELING, JA Hoorn (NL); Anubrata BHOWMICK, JM Den Helder (NL); Kasper TIEBIE, AJ Anna Paulowna (NL)

(73) Assignee: Quest Photonic Devices B.V., SR Wieringerwerf (NL)

(21) Appl. No.: 19/079,695

(22) Filed: Mar. 14, 2025

(30) Foreign Application Priority Data
Mar. 14, 2024 (EP) 24 163 574.7

(51) Int. Cl. A61B 5/00; G06T 5/70; G06V 10/143; G06V 10/25

(52) U.S. Cl. CPC A61B 5/0071; A61B 5/415; G06T 5/70; G06V 10/143; G06V 10/25; G06T 2207/10024; G06T 2207/10064; G06T 2207/10152; G06T 2207/20104; G06T 2207/20212; G06T 2207/30004; G06V 2201/031

(57) ABSTRACT

A method for visualization of faint fluorescence in surgery. The method including: emitting excitation light from an excitation light source onto an operation area containing a faint fluorescence source, as well as white light from a white light source, capturing one or more fluorescence images of the operation area at a wavelength range of fluorescence light emitted by the faint fluorescence source as well as one or more white light images, performing image processing on the one or more fluorescence images and creating one or more false color fluorescence images from the one or more fluorescence images, wherein a contrast between the fluorescence light emitted by the faint fluorescence source and background is increased, and creating one or more composite images by overlaying the one or more false color fluorescence images over the one or more white light images.

### CROSS-REFERENCE TO RELATED APPLICATION

The present application is based upon and claims the benefit to EP 24 163 574.7 filed on Mar. 14, 2024, the entire contents of which is incorporated herein by reference.

### BACKGROUND

### Field

The present disclosure relates to a method and a system for visualization of faint fluorescence in surgery, such as open surgery, as well as a software program.

### Prior Art

Fluorescence-guided surgery using fluorescent dyes is known in the art. Fluorescent dyes such as indocyanine green (ICG) or others are injected into tissue to be examined or operated on and, once the fluorescent dye has infiltrated the tissue, fluorescence excitation light is shone on the dye infused tissue, causing the fluorescent dye to emit fluorescence emission light, also called fluorescence light in short.

The basic principle of induced fluorescence is that the molecules making up fluorescent dyes have an excitation wavelength spectrum in which they are capable of capturing excitation light, absorbing the energy of the captured excitation light and transitioning to an excited state. The excited energy state of the fluorescent molecules is not stable. After a short period, excess energy is released from the molecule in the form of de-excitation light, also called fluorescence light leaving the fluorescent molecule once again in its basic energy state. The fluorescence light is slightly less energetic than the excitation light, which means that the fluorescent dyes' excitation spectrum is slightly shifted towards smaller wavelengths with respect to its fluorescence spectrum.

Therefore, excitation light used for exciting fluorescence in fluorescent dyes has a wavelength that is slightly offset towards smaller wavelengths than the fluorescence light that is to be observed. This is useful, since the amount of excitation light reflecting from the fluorescent dye infused tissue is orders of magnitude more intense than the resulting fluorescence light itself, and it would be impossible to detect fluorescence light in the presence of all the reflected excitation light. This slight shift in wavelength is exploited by using a wavelength filter configured to let light with the wavelength of the fluorescence light pass to shield off reflected excitation light from entering a camera.

Fluorescent dyes are not the only source of fluorescence in body tissues. Other sources include fluorescent probes used to label certain tissue types, such as cancerous tissues, and autofluorescent tissues such as the parathyroid. The parathyroid gland includes naturally occurring fluorescent molecules that emit fluorescence light in the near infrared spectrum when excited by excitation light of suitable wavelengths. However, compared with the above-described fluorescent dyes, the fluorescence signal emitted by autofluorescence is orders of magnitude weaker, i.e., orders of magnitude more prone to be outshone by excitation light or other sources such as ambient light having components in the spectral region of the autofluorescence.

The autofluorescence of the parathyroid is excited in a wavelength range around 785 nm and emits circa from 820 nm to 830 nm. This overlaps with, for instance, ICG, which emits in the range from 800 nm to 830 nm and is excited around 780 nm.

There are also autofluorescent colonic tissues and colonic cancers in the UV-and visible spectra, such as described for example in Li BH, Xie SS, “Autofluorescence excitation-emission matrices for diagnosis of colonic cancer,” World J Gastroenterol. 2005 Jul. 7;11(25):3931-4 or Bhaskar Banerjee M.D. et al., “Tryptophan autofluorescence imaging of neoplasms of the human colon,” J. Biomed. Opt. 17(1) 016003 (1 Feb. 2012).

The same holds for fluorescent probes used to label tissue types, such as cancerous tissues. Such probes and markers produce faint fluorescence signals of similar magnitude as the autofluorescence of the parathyroid gland, that is, orders of magnitude less than fluorescent dyes commonly used in fluorescence-guided surgery.

For these reasons, there is no fluorescence-guided surgery available that is based on faint fluorescence sources such as the autofluorescence of the parathyroid gland or fluorescent probes that image fluorescence light at a similar faint intensities as the aforementioned auto-fluorescence.

### SUMMARY

An object is to provide method and apparatus for utilizing faint fluorescence sources for fluorescence-guided surgery, such as open surgery.

Such object can be achieved with a method for visualization of faint fluorescence in surgery, such as open surgery, comprising:
emitting excitation light from an excitation light source onto an operation area containing a faint fluorescence source, such as parathyroid tissue exhibiting autofluorescence and/or fluorescent probes exhibiting fluorescence at similar intensity as the autofluorescence, as well as white light from a white light source,
capturing one or more fluorescence images of the operation area at a wavelength range of fluorescence light emitted by the faint fluorescence source as well as one or more white light images,
performing image processing on the one or more fluorescence images and creating one or more false color fluorescence images from the one or more fluorescence images, wherein a contrast between the fluorescence light emitted by the faint fluorescence source and background is increased, and
creating one or more composite images by overlaying the one or more false color fluorescence images over the one or more white light images.

The present method can be applicable for both still images and image sequences, such as video imagery. Increasing the contrast between the fluorescence light emitted by the faint fluorescence source and the background can help in making the faint fluorescence signal visible over the background, which can then be masked out.

The fluorescence images can be captured using an image sensor at high gain settings. Due to the high gain settings, the sensor can go into saturation in parts of the image with medium to high light intensity, whereas the darker regions can be enhanced and occupy the dynamic range of the picture. Still, the faint fluorescence signal can occupy the lower part of the dynamic spectrum.

In embodiments, the contrast between the fluorescence light emitted by the faint fluorescence source and background can be increased by applying an inverse gamma correction. A gamma correction is a nonlinear transformation of luminance values in video or still image systems. This nonlinear transformation can darken relatively dark areas in the image, whilst relatively light areas of the image can keep their relatively light intensity.

However, in the present case, it is the dark parts of the fluorescence images that can be contrast-enhanced, and therefore the inverse exponent of the logarithmic function can be used, hence the name “inverse gamma correction”. The inverse gamma correction may also be called decoding gamma or gamma expansion.

Depending on the setup and on the nature of the fluorescence light, the white light images and the fluorescence images can be captured either simultaneously or alternately in embodiments. The simultaneous capturing of white light images and fluorescence images can be practicable in case that the fluorescence signal lies outside of the visible light spectrum captured in the white light images and an image capturing device is available that comprises image sensors that are sensitive in the white light on the one hand and in the region of the fluorescence signal on the other hand.

If, on the other hand, the fluorescence light has a wavelength that falls within the white light spectrum, the white light images and the fluorescence images cannot be captured simultaneously since the white light illumination would drown out the fluorescence signal. In such cases, it is possible to alternate between white light illumination and fluorescence illumination and to use pairs of a white light image and an immediately subsequently captured fluorescence light image, or vice versa, to provide the composite images.

It is also possible to interject a fluorescence image capturing once in a while in between otherwise uninterrupted sequences of white light images and to use the captured fluorescence image to be overlaid over each of the sequence of white light images, such as if the sequence of white light images does not undergo rapid movements or such movements are identified and the location of the intermittent fluorescence image is adjusted to conform to the movement to be overlaid over the correct spot in each of the white light images. Such intermittent capturing of fluorescence images can have the added benefit that there is less flicker in the illumination of the operating area. Movements can be offset by the methods described in relation to the realignment of the region of interest.

In embodiments, the image processing on the one or more fluorescence images can comprise at least one of masking specular reflection, noise suppression and normalization. Specular reflections can show up as saturated regions of the images, either in the white light images, the fluorescence images or both. Such saturated regions can be excluded from further image processing in the fluorescence images and masked as background, for example by setting the brightness value of the afflicted pixels of the fluorescence image to zero, that is, black.

Noise suppression is a known technique in image processing and beneficial in the present context, since the fluorescence signals from faint fluorescence sources can be very weak. Such weak signals have a low signal to noise ratio so that it might be difficult to sort out the faint fluorescence signal from the noise, depending on circumstances. Known noise suppression techniques are filtering over the adjacent pixels and taking mean values of the brightness of adjacent pixels or pixel clusters. Known removal algorithms reduce or remove the visibility of noise by smoothing the entire image, leaving areas near contrast boundaries. These methods can obscure fine, low contrast details, but in the present context, where the goal is to identify structures and tissues exhibiting faint fluorescence, this trade-off can be used in order to receive a clear signal identifying the structure or tissue in the first place.

The normalization performed during image processing of the one or more fluorescence images can be used in order to create the false color images representing the fluorescence images. Normalization means that the dynamic range of a fluorescence image is focused on the interesting part of the fluorescence image, which in the case of faint fluorescence sources is the low intensity part of the fluorescence image. In embodiments, the normalization can comprise a percentile normalization, such as comprising cutting off the darkest 1 to 2% and the brightest 1 to 2% of the pixels of the fluorescence image, which removes saturated areas as well as background areas without signal that only contain noise. Alternatively or in addition, the normalization can comprise selecting a bottom part of the brightness spectrum and spreading the brightness information contained therein over the full brightness spectrum, wherein the bottom part of the brightness spectrum can include 0% to 20%, 0% to 10%, 0% to 5%, of the brightness spectrum of the input fluorescence images. The extent of the chosen region can be adapted to the actual gain settings and signal distributions observed under the specific circumstances of the surgery.

The threshold below which the faint fluorescence signal is found and above which the signal should be discarded may also be adjusted automatically or manually by the surgeon or an assistance to the surgeon in order to gain optimal visibility of the faint fluorescence signal in the composite image. Automatic adjustment of the threshold may be achieved by automatic analysis of the signal amplitude distribution. For example, the normalization algorithm may start with a threshold of 30% of the maximum of the brightness of the raw fluorescence image and select an isolated spot in the fluorescence image with the highest intensity below that threshold and select a new threshold representing a brightness value just above that relatively brightest spot. By selecting an isolated spot it can be avoided that the algorithm simply arrives at the border to a saturated region that is being masked out using the initial 30% threshold. If, however, there are other isolated spots that go into saturation using the lower threshold, the threshold can be increased again until all isolated spots are shown without their centers going into saturation, i.e., the brightness in the center of the respective spot being greater than the threshold.

In embodiments, the image processing can comprise creating a virtual region of interest in the white light image and/or the fluorescence image, wherein the fluorescence image can be cut off outside the region of interest.

By creating a virtual region of interest and cutting off the fluorescence image outside of this region of interest, the fluorescence can only be visible inside the region of interest, while outside the region of interest the composited imagery can consist only of the white light image. This can make it much easier for a surgeon to identify the faint fluorescence source, as fluorescence light from outside of the region of interest does not interfere with the fluorescence light from the faint fluorescence source. The intensity of the false color fluorescence image can be normalized according to the intensity distribution inside the region of interest. Normally, the fluorescence light from the faint fluorescence source may easily be outshone by excitation light or other sources such as ambient light having components in the same spectral region. By limiting the visibility of light in this spectral region to the region of interest, these sources can be largely suppressed, resulting in a much larger contrast of the faint fluorescence source.

Furthermore, the brightness of fluorescence from the faint fluorescence source inside the region of interest may be used to adjust the gain of an imaging sensor, which might otherwise be set too low because of possible presence of specular reflections outside of the region of interest. Such bright spots outside the region of interest can be ignored, thus focusing the control of sensor gain and/or image processing on the region of interest containing the sought-after faint fluorescence signal.

The images may be still images or videos. By using videos for both the white light images and the fluorescence images, a video feed can be provided, in which the region of interest containing the parathyroid tissue or fluorescent probes is highlighted by providing fluorescence information on top of the white light video feed.

The region of interest can be created by using manual control elements in order to control the placement and/or shape of the region of interest. The manual control elements may be buttons, switches and/or a touchscreen display. With the help of these manual control elements, the surgeon may select the region of interest. For example, the surgeon may view a white light image of the operation area and select the region of interest based on this white light image. The surgeon may instead view a composite image consisting of an overlay of the false color fluorescence image over the white light image and select the region of interest based on this composite images or the surgeon may select the region of interest solely based on the fluorescence image. The region of interest may have any shape and form, for example a circle, an ellipse, a rectangular shape or a shape conforming to the contours of an organ or tissue visible in the white light image.

The region of interest can be automatically repositioned after a movement, in order to realign the region of interest in relation to the faint fluorescence source in the one or more composite images. The movement may be a movement of the patient or a movement of an image capturing device used to capture the images. Such movements may occur accidently during surgeries, for example due to a slight shift of the patient or shaking of the image capturing device. However, such movements may change the position of the region of interest with respect to the faint fluorescence source. This is undesirable, as it decreases the visibility of the fluorescence in the composite image and may hinder the surgeon during the surgery. To offset this movement, the region of interest can be automatically realigned to the faint fluorescence source. In this way, the movement does not impede the surgery.

According to another embodiment, the region of interest can be automatically repositioned by an algorithm, which can be trained to recognize a shape of an organ comprising or consisting of the faint fluorescence source, wherein the region of interest can be realigned in relation to the organ. The algorithm may be an artificial neural network, which has been trained with white light image data of organs, for example parathyroid glands, in order to reliably detect these organs in new white light images. Afterwards, the region of interest can be realigned in relation to the organ in order to offset some movement.

According to another embodiment, the region of interest can be automatically repositioned by performing image analysis on the white light image in order to identify an image region with a predominantly red wavelength spectrum, wherein the region of interest can be realigned in relation to or set as the image region with a predominantly red wavelength spectrum. In surgery, the patient is typically covered by a blanket or cloth, with just a small opening around the area of operation. The incision made in open surgery is typically visible as a red region in white light image, in contrast to the surrounding skin and the blanket or cloth. This can allow an identification of the incision by image analysis of the white light image, identifying the incision as the region with a predominantly red wavelength spectrum. Afterwards, the region of interest may be realigned such that it is centered on the region with a predominantly red wavelength spectrum or that it is set to equal the region with a predominantly red wavelength spectrum. This can be an easy way to offset a movement during surgery and mask any light in the relevant wavelength spectrum from outside of the incision.

According to an embodiment, specular reflections can be detected using high intensity information in the white light images, wherein areas comprising the specular reflections can be deleted from the region of interest. These areas can also reflect fluorescence light, such near infrared light, which can cause high intensity spots to appear in the fluorescence image, which is unwanted. Thus, these regions can be deleted from the region of interest, removing the fluorescence information in these areas from the composite image.

Such object can be further achieved by a system for visualization of faint fluorescence in surgery, such as open surgery, comprising:
a controller comprising an image processor,
a light source configured to produce excitation light and white light,
an image capturing device comprising one or more image sensors configured to capture fluorescence images and white light images,
wherein the controller is configured to control the operation of the light source and the image capturing device and to carry out the method according to one of the previously described embodiments.

The image capturing device may be configured as a camera. The image sensors may comprise first image sensor for the white light and a second image sensor for the fluorescence light. The controller may be a computer. The controller can be configured to control the light source in order to emit the excitation light and the white light onto the operation area and to control the image capturing device in order to capture the one or more fluorescence images and the one or more white light images. The image processor can perform the image processing, such as the creation of the region of interest and/or the creation of the false color fluorescence image and/or the creation of the composite images.

The light source can comprise an excitation light source and a white light source. The white light source may be a light source having no emission within the spectral range of the fluorescence signal of the faint fluorescence source, such as, for example, LEDs or laser sources. The system can comprise a lighting device and image capturing device, configured to emit the white light and the excitation light and to capture the white light image and the fluorescence image, respectively. Thus, the emission of the light and the capturing of the images is combined in a single device.

Such object can be additionally achieved by a software program configured to execute the method according to one of the previously described embodiments when run on the controller of the system according to one of the previously described embodiments.

The system for visualization of faint fluorescence in surgery and the software program embody the same advantages, features and characteristics as the method described above. Features mentioned with respect to the method are expressly applicable to the system and vice versa, as well as to the software program performing the control of the system components and the image processing part of the method, as well as receiving input from system components.

### BRIEF DESCRIPTION OF THE DRAWINGS

Further characteristics will become apparent from the description of the embodiments together with the claims and the included drawings. Embodiments can fulfil individual characteristics or a combination of several characteristics.

The embodiments are described below, without restricting the general intent of the invention, based on exemplary embodiments, wherein reference is made expressly to the drawings with regard to the disclosure of all details that are not explained in greater detail in the text. In the drawings:

FIG. 1 illustrates a schematic simplified representation of a system for visualization of faint fluorescence in a surgery,

FIG. 2 illustrates a schematic simplified representation of another system for visualization of faint fluorescence in a surgery,

FIG. 3 illustrates a schematic simplified representation of a composite image of an operation area, consisting of an overlay of a white light image and a fluorescence image, and

FIG. 4 illustrates a schematic simplified representation of a method for visualization of faint fluorescence in surgery.

In the drawings, the same or similar types of elements or respectively corresponding parts are provided with the same reference numbers in order to prevent the item from needing to be reintroduced.

### DETAILED DESCRIPTION

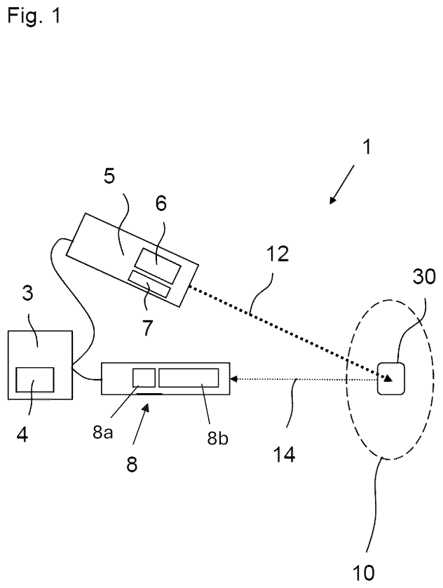

FIG. 1 shows a schematic simplified representation of a system 1 for visualization of faint fluorescence in a surgery. The system 1 comprises a controller 3 comprising hardware with an image processor 4 implemented as either image processing software on a general purpose computer or as a dedicated image processor configured to process the imagery. The controller 3 is connected to and controls a light source 5 and an image capturing device 8 comprising one or more image sensors 8a, such as a CCD or CMOS sensor, as well as optics, electronics, filters and the like (collectively represented in FIG. 1 as 8b). The light source 5 comprises both an excitation light source 6 and white light source 7 to illuminate an operation area 10. Excitation light 12 emitted by the light source 5 stimulates autofluorescent tissue 30 in the operation area 10 or fluorescent probes exhibiting fluorescence of similar intensity as the autofluorescence. The autofluorescent tissue 30, for example parathyroid tissue, will emit fluorescence light 14 at a specific wavelength range. This wavelength range will typically be in the near infrared spectrum and will usually have no overlap with the white illumination light in order to separate the white light from the fluorescence light 14.

The image capturing device 8 can be configured to capture imagery at this wavelength range as well as white light imagery. Typically, the image capturing device 8 can be configured with a first image sensor for white light, for example a RGB unit, and a second image sensor for the fluorescence light 14. The imagery captured by the image capturing device 8 is transmitted to the image processor 4 of the controller 3. The image processor 4 performs image processing in order to enhance the visibility of specific features of interest in the operation area 10.

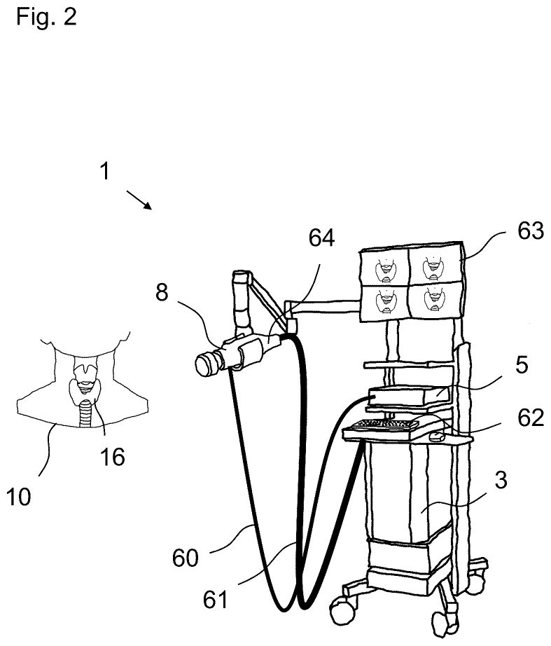

FIG. 2 shows another embodiment of a system 1 for visualization of faint fluorescence in an open surgery. In this embodiment, the image capturing device 8 and the lighting are combined to a single lighting and image capturing device 64. The device 64 is connected via a fiber cable 60 to the light source 5 in order to illuminate the operation area 10 of the parathyroid. The operation area 10 comprises a faint fluorescence source 16, e.g. the autofluorescent parathyroid gland. The image capturing device 8 captures a white light image and a fluorescence image of the operation area 10 and transmits the image information via data cable 61 to the controller 3, for example an industrial computer. The images may be displayed on the display 63. Manual control elements 62, for example a keyboard and a mouse, may be used by a surgeon to select a region of interest 20 in the images and/or to perform image processing. The image processing may comprise increasing a contrast between a signal from the faint fluorescence source 16 and a background, applying an inverse gamma correction, masking specular reflections, applying noise suppression and/or applying normalization.

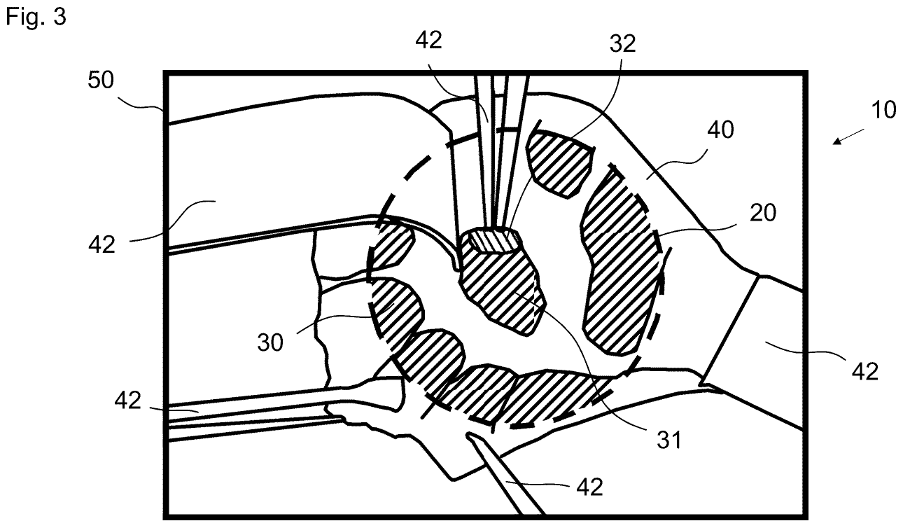

FIG. 3 shows a schematic simplified representation of a composite image 50 created by the image processor 4. The composite image 50 may be displayed on display 63 and consists of an overlay of a false color fluorescence image over a white light image. The operation area 10 shown in the composite image 50 comprises tissue 40 and several surgical tools 42 used during an open surgery. Fluorescence is represented by dashed lines in FIG. 3. In the center of the image, an autofluorescent organ 31, for example a parathyroid gland, is shown with a specific part emitting a particular fluorescent signal 32 of higher intensity than the fluorescence of the other autofluorescent tissue 30. Still, the intensity of the fluorescent signal 32 is much smaller than the intensity of the excitation light or other sources of light such as ambient light having components in the spectral region of the autofluorescence.

In order to enhance the contrast in the composite image 50, the surgeon may create or select a region of interest 20 in the image. The region of interest 20 is represented as a dashed circle in FIG. 3, however it may have a different form, location and/or size. Outside of the region of interest 20, only the white light image is shown in the composite image 50, while the fluorescence image is cut off. The surgeon may select and place the region of interest 20 using the manual control elements 62 or select the region of interest 20 directly on the display 63 showing the composite image 50.

In case that the patient or the image capturing device 8 moves during the surgery, the region of interest 20 may no longer align with the organ 31. In order to offset such a movement, the system 1 is configured to track the movement and realign the region of interest 20 accordingly. In order to track the movement, the image processor 4 may perform shape detection on high intensity information in the white light image in order to detect specular reflection. Specular reflection will typically result in intensity spikes in the white light image, which are used to identity certain regions in the image and help correct the positioning of the region of interest 20. The specular reflections may also be detected by the image capture unit for the fluorescence light 14. In addition, the image processor 4 may utilize an algorithm trained to recognize the shape of the organ 31, e.g. the parathyroid, in order to correct the positioning of the region of interest 20. Also, the image processor 4 may position the region of interest 20 based on the colors in the white light image. Specifically, as tissue in an open surgery typically appears red in a white light image, the region of interest 20 is placed on a region predominantly in the red wavelength range.

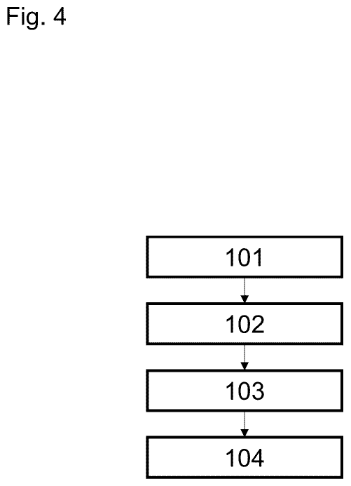

FIG. 4 shows a schematic simplified representation of method steps of a method for visualization of faint fluorescence in surgery. At step 101, excitation light 12 is emitted from the excitation light source 6 onto the operation area 10 containing the faint fluorescence source, for example parathyroid tissue exhibiting autofluorescence. At step 102, one or more images of the operation area 10 at a wavelength range covering the wavelength range of the faint fluorescence source as well as one or more white light images are captured. At step 103, image processing is performed on the captured images. The image processing may include the creation of a virtual region of interest 20 in the white light image and/or the fluorescence image. Outside the region of interest 20, any image information of the fluorescence image is cut off. Image processing may also include increasing a contrast between the faint fluorescence source 16 and a background, applying an inverse gamma correction, masking specular reflections, applying noise suppression and/or applying normalization. At step 104, a false color fluorescence image visible in the visible light spectrum is created from the fluorescent image. Then, at step 105, a composite image 50 is created by overlaying the false color fluorescence image over the white light image. In this composite image 50, the false color fluorescence is only visible inside the region of interest 20.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### List of References

1 System
3 controller
4 image processor
5 light source
6 excitation light source
7 white light source
8 image capturing device
8a one or more image sensors
8b optics, electronics, filters etc.
10 operation area
12 excitation light
14 fluorescence light
20 region of interest
30 autofluorescent tissue
31 autofluorescent organ
32 fluorescence signal
40 tissue
42 surgical tool
50 composite images
60 fiber cable
61 data cable
62 control elements
63 display
64 light source and image capturing device
101-104 method steps

What is claimed is:

1. A method for visualization of faint fluorescence in surgery, the method comprising:
emitting excitation light from an excitation light source onto an operation area containing a faint fluorescence source, as well as white light from a white light source,
capturing one or more fluorescence images of the operation area at a wavelength range of fluorescence light emitted by the faint fluorescence source as well as one or more white light images,
performing image processing on the one or more fluorescence images and creating one or more false color fluorescence images from the one or more fluorescence images, wherein a contrast between the fluorescence light emitted by the faint fluorescence source and background is increased, and
creating one or more composite images by overlaying the one or more false color fluorescence images over the one or more white light images.

2. The method according to claim 1, wherein the fluorescence images are captured using an image sensor at high gain settings.

3. The method according to claim 1, wherein the contrast between the fluorescence light emitted by the faint fluorescence source and background is increased by applying an inverse gamma correction.

4. The method according to claim 1, wherein the white light images and the fluorescence images are captured one of simultaneously or alternately.

5. The method according to claim 1, wherein the image processing on the one or more fluorescence images comprises at least one of masking specular reflection, noise suppression and normalization.

6. The method according to claim 5, wherein the normalization comprises a percentile correction.

7. The method according to claim 6, wherein the percentile correction comprises one or more of:
cutting off the bottom 1 to 2% and the top 1 to 2% of the pixels of the fluorescence image, and
selecting a bottom part of the brightness spectrum and spreading the brightness information contained therein over the full brightness spectrum.

8. The method according to claim 7, wherein the bottom part of the brightness spectrum includes one of 0% to 20%, 0% to 10%, or 0% to 5%, of the brightness spectrum of the input fluorescence images.

9. The method according to claim 1, wherein the image processing comprises creating a virtual region of interest in one or more of the white light image and the fluorescence image, wherein the fluorescence image is cut off outside the region of interest.

10. The method according to claim 9, wherein the region of interest is created by using manual control elements in order to control one or more of the placement and shape of the region of interest.

11. The method according to claim 9, further comprising automatically repositioning the region of interest after a movement, in order to realign the region of interest in relation to the faint fluorescence source in the composite images.

12. The method according to claim 11, wherein the automatically repositioning is performed by an algorithm, which is trained to recognize a shape of an organ comprising the faint fluorescence source, wherein the region of interest is realigned in relation to the organ.

13. The method according to claim 11, wherein the automatically repositioning is performed by image analysis on the white light image in order to identify an image region with a predominantly red wavelength spectrum, wherein the region of interest is realigned in relation to or set as the image region with a predominantly red wavelength spectrum.

14. The method according to claim 9, wherein specular reflections are detected using high intensity information in the white light images, wherein areas comprising the specular reflections are deleted from the region of interest.

15. The method according to claim 1, wherein the excitation light source comprises one or more of parathyroid tissue exhibiting autofluorescence and fluorescent probes exhibiting fluorescence at similar intensity as the autofluorescence.

16. A system for visualization of faint fluorescence in surgery, the system comprising:
a controller comprising an image processor comprising hardware,
a light source configured to produce excitation light and white light,
an image capturing device comprising one or more image sensors configured to capture fluorescence images and white light images,
wherein the controller is configured to control the operation of the light source and the image capturing device and to:
emit excitation light from an excitation light source onto an operation area containing a faint fluorescence source, as well as white light from a white light source,
capture one or more fluorescence images of the operation area at a wavelength range of fluorescence light emitted by the faint fluorescence source as well as one or more white light images,
perform image processing on the one or more fluorescence images and creating one or more false color fluorescence images from the one or more fluorescence images, wherein a contrast between the fluorescence light emitted by the faint fluorescence source and background is increased, and
create one or more composite images by overlaying the one or more false color fluorescence images over the one or more white light images.

17. The system according to claim 16, wherein the one or more image sensors comprising a first image sensor configured to capture the one or more white light images and a second image sensor configured to capture the one or more fluorescent images.

18. A non-transitory computer-readable storage medium storing instructions that cause a computer to at least perform:
emitting excitation light from an excitation light source onto an operation area containing a faint fluorescence source, as well as white light from a white light source,
capturing one or more fluorescence images of the operation area at a wavelength range of fluorescence light emitted by the faint fluorescence source as well as one or more white light images,
performing image processing on the one or more fluorescence images and creating one or more false color fluorescence images from the one or more fluorescence images, wherein a contrast between the fluorescence light emitted by the faint fluorescence source and background is increased, and
creating one or more composite images by overlaying the one or more false color fluorescence images over the one or more white light images.
